(19) United States
(12) Patent Application Publication
(10) Pub. No.: US 2024/0390733 A1
(43) Pub. Date: Nov. 28, 2024

(54) PORTABLE NETWORKED EXERCISE BALL-AND-STICK APPARATUS

(71) Applicant: Total Motion 360, Port Jefferson, NY (US)

(72) Inventor: Tom Fabbri, Port Jefferson, NY (US)

(21) Appl. No.: 18/648,210

(22) Filed: Apr. 26, 2024

Related U.S. Application Data
(60) Provisional application No. 63/462,125, filed on Apr. 26, 2023.

(51) Int. Cl. A63B 24/00; A63B 43/00; A63B 43/02; G16H 40/67

(52) U.S. Cl. CPC A63B 24/0062; A63B 43/004; A63B 43/02; G16H 40/67; A63B 2024/0068; A63B 2024/0071; A63B 2220/803; A63B 2220/833; A63B 2225/20; A63B 2225/50

(57) ABSTRACT

The present invention is a connected portable exercise apparatus that allows for weight resistance or weight-bearing exercise. A portable exercise ball-and-stick apparatus, which includes a base having a partial and concave spherical region for accepting therein a ball and with a rod-portion extending from the ball, which may be either linearly- or non-linearly shaped, and preferably with a handle at the end of the rod distal from the ball for gripping the rod with the handle preferably being rotatable relative to the rod. The handle has a sensor in it that allows for the determination of the motion of the rod. The motion of the rod is compared to desired movements in a given exercise, and feedback is given to the user when they go outside of the desired movements for an exercise.

### CROSS-REFERENCE TO RELATED APPLICATIONS

The present patent application claims the priority benefit of U.S. provisional patent application No. 63/462,125 filed Apr. 26, 2023, the disclosure of which is incorporated by reference herein.

### BACKGROUND OF THE INVENTION

### Field of the Disclosure

The present disclosure is generally related to receiving and processing data from sensors associated with a portable exercise apparatus that may readily be used in a home or office environment to provide feedback during or after exercise sessions.

### Description of the Related Art

Generally, it is known that there are numerous exercise devices for home use or use outside of a formal gymnasium setting, including “use at home” weight-lifting devices, such as sets of barbells and other weight-resistant exercises. Most of these types of home use weight-resistance or weight-strengthening devices may not be readily movable without great difficulty, generally requiring professional moving services.

Moreover, due to the differences in body types (e.g., height, weight, strength, flexibility) and fitness goals across different users, the same equipment may need to be configured and/or used differently in order to achieve maximum effect and progress towards the desired fitness goals. Many users, however, lack the knowledge and background on how to effectively create and implement exercise regiments for their respective body types and fitness goals, which may thus result in lack of progress, user frustration, and/or injury.

Therefore, there is a need in the art for improved systems and methods of providing for portable smart exercise apparatuses that allow users to train effectively without being tied to cumbersome equipment or expensive gyms or personal trainers.

### SUMMARY OF THE CLAIMED INVENTION

Embodiments of the present invention may provide for a portable exercise apparatus that includes a base having a partial and concave spherical region for accepting therein a ball and with a rod-portion extending from the ball that may be either linearly or non-linearly-shaped. The apparatus may preferably also include a handle at the end of the rod distal from the ball for gripping the rod. The rod-whether linear or non-linear—may be preferably made of a metal (e.g., iron or steel) of sufficient density for providing considerable weight to the rod. The handle at the end of the rod distal from the ball may be preferably made to be rotatable vis-à-vis the rod, thus allowing a user to rotate and otherwise manipulate the rod without having to alter the user's original grip of the handle.

In some embodiments, the apparatus may be part of or incorporated into smart workout equipment that allow a user to recreate both the gym and fancy studio classes at home with live and on-demand classes, touchscreen displays, built-in cameras, and all-in-one systems, and having personal or AI-generated guidance for managing workouts so as to optimize progress toward a user's specific fitness goal (or goals).

### BRIEF DESCRIPTIONS OF THE DRAWINGS

FIG. 1 illustrates an exemplary network environment in which a portable networked exercise ball-and-stick apparatus may be implemented.

FIG. 2 illustrates an exemplary exercise database.

FIG. 3 is a flowchart illustrating an exemplary method for exercise analytics.

FIG. 4 is a flowchart illustrating an exemplary method for apparatus configuration.

FIG. 5 is a flowchart illustrating an exemplary method for feedback-based exercise management.

FIG. 6 is a block diagram of an exemplary computing device that may be used to implement an embodiment of the present invention.

### DETAILED DESCRIPTION

Embodiments of the present disclosure will be described more fully hereinafter with reference to the accompanying drawings in which like numerals represent like elements throughout the several figures and in which example embodiments are shown. Embodiments of the claims may, however, be embodied in many different forms and should not be construed as limited to the embodiments set forth herein. The examples set forth herein are non-limiting examples and are merely examples among other possible examples.

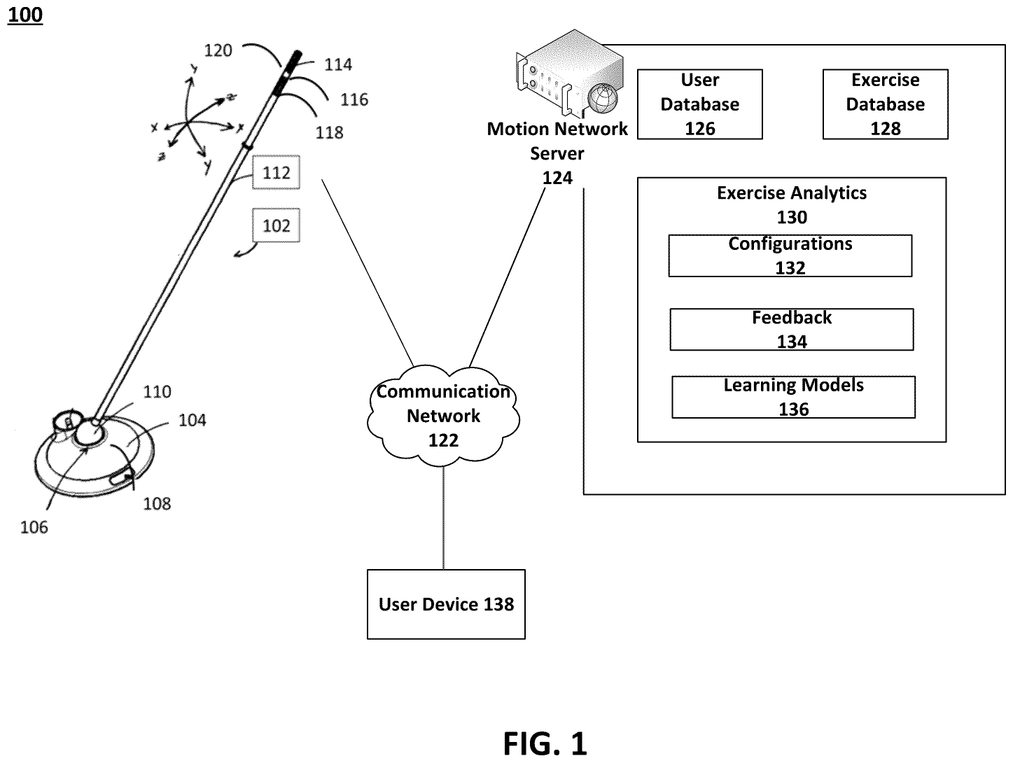

FIG. 1 illustrates an exemplary network environment 100 in which a portable networked exercise ball-and-stick apparatus 102 may be implemented. The apparatus 102 includes a base 104 with ball 110 located in a partial and concave spherical region 106 and associated with a base sensor 108. A stick or rod 112 may be affixed to ball 110 and extend vertically from the fixation point. Rod 112 preferably includes a handgrip or handle 114 for a user to grip the upper portion of rod 112 when exercising.

Ball 110 sits in the concave spherical region 106 of base 104 so that rod 112 is freely movable when seated in the concave spherical region 106 of the base 104. The rod 112 is, preferably, about five to seven feet in length and weighs forty or fifty pounds, though the invention is not limited to a rod having these physical characteristics. The rod 112 is preferably made of dense steel, iron, or other metal or material of sufficient weight to provide meaningful weight resistance for exercising. A user is able to move rod 112 either from side-to-side or in any direction, without limitation, since ball 110 uninhibitedly sits within the concave spherical region 106 of the base 104. Other means for rendering the handle rotatable relative to the rod so that a user does not need to readjust his grip during the use of the inventive apparatus are also within the scope of the claimed invention.

The ball 110 is preferably made of high-density steel, iron, or similar metal. In use, the ball is to be freely rotatable within the partial, concave spherical region of the base so that the user may lift the rod from either its handgrip portion or at a part of the rod that is more intermediate between the ball and the distal end of the rod preferably having the handle, thereby effectively increasing the weight that the user bears. The user may also “toss” the rod from one hand to the other as a manner of exercise. When in use, the ball rests within the partial, concave spherical region of the base but is not itself attached to the base so that it (and the attached rod) is freely rotatable by the user for permitting exercise.

The base 104 having the partial, concave spherical region 106 for supporting the ball 110 in a freely rotatable position may, preferably, be made of either a plastic material or a metal, such as titanium, and should be of both sufficient durability to support the weight of the ball 110 and rod 112 attached to the ball, yet still be of a weight that is sufficiently light for permitting the user to readily grasp and carry the base to another location. It is further preferable that the base include either a separate handle portion for more easily allowing it to be carried by a person or, in the alternative, one or more cutout portions near the perimeter of the base for use as one or more handles for readily transport by a single person. Preferably, the rod 112 is permanently affixed to the ball 110, though this is not required, and the rod 112 may instead be removably attached to the ball 110, for example, by way of a threaded-screw connection, a snap-in connection, etc.

The concave spherical region 106 of base 104 may have a base sensor 108 that may detect the presence and orientation of ball 110. In one embodiment, the base sensor 108 may be a differential pressure sensor that allows for measuring pressure differences between two or more pressure values under the ball 110. In one embodiment, the base sensor 108 may be a differential pressure sensor that allows for measuring pressure differences between two or more pressure values under the ball 110. The pressure difference may be used by the feedback module 134 to determine the position of the rod 112 and handle 114. The base sensor 108 may be an optical sensor that may track movement or markings on the ball 110 to determine the position of the rod 112 and handle 114. The base sensor 108 may be an optical sensor that tracks the user's position or the rod 112. In embodiments with an optical sensor tracking the user's movements, the base sensor 108 would be located in the base 104 but not in the concave spherical region 106.

The handle 114 may be a rubber or foam grip that contains one or more handle sensors 116 and a radio 118. The handle sensor 116 may be an accelerometer to allow the feedback module 134 to monitor the movement of the rod 112 and determine the relative position of the handle during an exercise. In one embodiment, there may be more than one handle sensor 116. These may include a pressure sensor to determine when and how a user is gripping the handle. Other sensors may be used to determine the user's status, such as their body temperature, pulse rate, respiration rate, pulse oxygen, etc. Other sensors may also determine the status of the exercise environment, such as the humidity, temperature, etc. The handle sensor 116 or base sensor 108 may be a microphone to allow the user to interact through voice prompts and a natural language processor.

The handle 114 may have a radio 118 to connect to the communication network (or cloud) 122 and transmit positional data from the handle sensor 116 and or the base sensor 108 to the motion network server 124. The radio may allow connection to a cellular network. It may also allow for connections via Wi-Fi, Bluetooth, or ZigBee. The radio 118 may connect to a user device 138, such as a smartphone, in some embodiments. The handle 114 may have a vibrator 120, which can provide haptic feedback when performing an exercise outside of the desired movement parameters. Further, embodiments may include a cloud 122 or a communication network 122 that may be a wired and/or a wireless network.

The communication network 122, if wireless, may be implemented using communication techniques such as visible light communication (VLC), worldwide interoperability for microwave access (WiMAX), long term evolution (LTE), wireless local area network (WLAN), infrared (IR) communication, public switched telephone network (PSTN), radio waves, or other communication techniques that are known in the art. The communication network 122 may allow ubiquitous access to shared pools of configurable system resources and higher-level services that can be rapidly provisioned with minimal management effort, often over the internet, and relies on sharing resources to achieve coherence and economies of scale, like a public utility. In contrast, third-party clouds allow organizations to focus on their core businesses instead of expending resources on computer infrastructure and maintenance.

The cloud 122 may be communicatively coupled to the exercise apparatus 102 to the motion network server 124. The motion network server 124 may receive movement data from the handle sensor 116 and or base sensor 108 and provide feedback to the user indicating if they are performing a given exercise incorrectly by delivering haptic feedback through the vibrator 120 when the user moves the handle outside of the prescribed movement path of a given exercise.

Embodiments may include a user database 126, which may store information about users of the motion network server 124. This may include account information such as email address, user device 138 identification, exercise apparatus 102 identifications, etc. The user database 126 may also store exercise information about the user. Exercise information may include information about the user's build that would impact the correct exercise motion. For example, the correct range of motion for a squat will be different in a five feet tall user and another user who is six and a half feet tall. Exercise information may also include historical exercise information from a user. This historical information may include what exercises a user had done when they were done, the duration of the exercise(s), and the accuracy of the exercise movements.

Embodiments may include an exercise database 128, which may store the desired movement patterns of exercises. For example, the exercise tall kneeling press has the desired movement range that includes at least two feet of vertical movement with no more than one inch of lateral movement. In one embodiment, the amount of desired vertical movement is based on the user's height.

Embodiments may include an exercise analytics engine 130, which allows the user to input information through the configuration module 132 and prompt the feedback module 134 when the user is engaged in an exercise, which may be used to analyze the exercise movements and generate custom feedback to the user in real-time. Exercise analytics engine 130 may include data and algorithms associated with generating and customizing configurations 132, feedback 134, and learning models 136.

The configurations 132 may be used to configure a workout program for a particular user (e.g., having particular demographics or parameters). The information collected from the user may include entering data about their height weight and build into the user database 126. The user data may include exercise history, plans, and fitness goals in some embodiment. Feedback 134 may include algorithms executable to monitor the movement of the handle sensor 116 or base sensor 108 to determine if the user is performing an exercise properly. Finally, learning models 136 may be developed for specific users or groups of users having similar physical parameters (e.g., height, weight, strength level, flexibility level, health conditions, etc.), exercise styles or preferences, and health goals. Such learning models 136 may correlate such factors to specific types of exercise or exercise parameters, thereby allowing for generation of custom exercise regimens for optimization of progress toward specific goals. Such regimens may further be adjusted in real-time as different users may diverge in terms of success rates, pace of progress, or lack thereof. Such feedback regarding the different user progress over time may be used as feedback to refine the learning models 136 and further tailor the correlations, models, and custom regimens to the user.

Further, embodiments may include a user device 138 such as a computing device, laptop, smartphone, tablet, computer, smart speaker, or I/O devices. I/O devices may be present in the computing device. Input devices may include but are not limited to keyboards, mice, trackpads, trackballs, touchpads, touch mice, multi-touch touchpads and touch mice, microphones, multi-array microphones, drawing tablets, cameras, single-lens reflex cameras (SLRs), digital SLRs (DSLRs), complementary metal-oxide-semiconductor (CMOS) sensors, accelerometers, infrared optical sensors, pressure sensors, magnetometer sensors, angular rate sensors, depth sensors, proximity sensors, ambient light sensors, gyroscopic sensors, or other sensors. Output devices may include but are not limited to video displays, graphical displays, speakers, headphones, etc. Devices may include but are not limited to a combination of multiple input or output devices such as Microsoft KINECT, Nintendo Wii remote, Nintendo WII U GAMEPAD, or Apple iPhone. Some devices allow gesture recognition inputs by combining input and output devices.

Other devices allow for facial recognition, which may be utilized as an input for different purposes such as authentication or other commands. Some devices provide for voice recognition and inputs, including, but not limited to, Microsoft KINECT, SIRI for iPhone by Apple, Google Now, or Google Voice Search. Additional user devices have both input and output capabilities, including, but not limited to, haptic feedback devices, touchscreen displays, or multi-touch displays. Touchscreen, multi-touch displays, touchpads, touch mice, or other touch sensing devices may use different technologies to sense touch, including but not limited to capacitive, surface capacitive, projected capacitive touch (PCT), in-cell capacitive, resistive, infrared, waveguide, dispersive signal touch (DST), in-cell optical, surface acoustic wave (SAW), bending wave touch (BWT), or force-based sensing technologies. Some multi-touch devices may allow two or more contact points with the surface, allowing advanced functionality including, but not limited to, pinch, spread, rotate, scroll, or other gestures. Some touchscreen devices, including, but not limited to, Microsoft PIXELSENSE or Multi-Touch Collaboration Wall, may have larger surfaces, such as on a table-top or on a wall, and may also interact with other electronic devices.

Some I/O devices, display devices, or groups may be augmented reality devices. An I/O controller may control one or more I/O devices, such as a keyboard and a pointing device, or a mouse or optical pen. Furthermore, an I/O device may also contain storage and/or an installation medium for the computing device. In some embodiments, the computing device may include USB connections (not shown) to receive handheld USB storage devices. In further embodiments, an I/O device may be a bridge between the system bus and an external communication bus, e.g., USB, SCSI, Fire Wire, Ethernet, Gigabit Ethernet, Fiber Channel, or Thunderbolt buses.

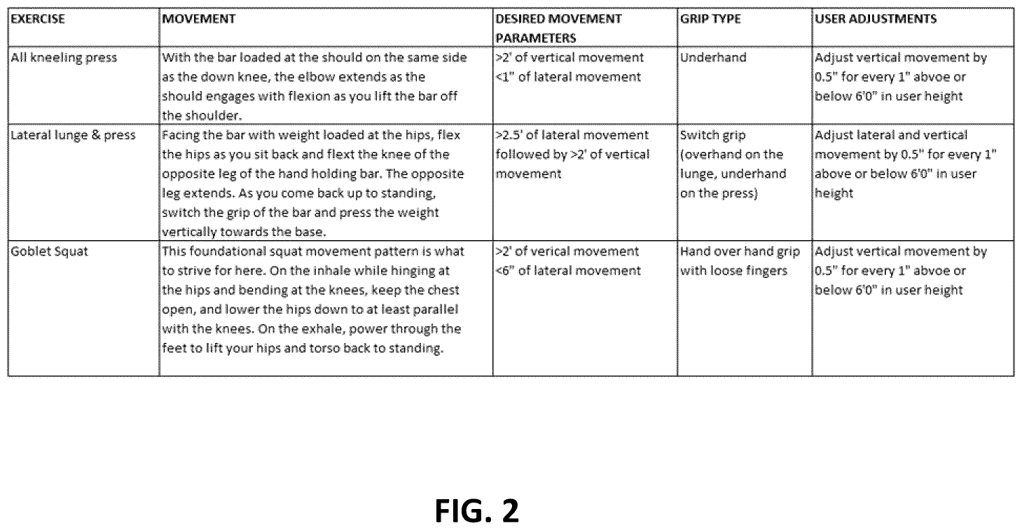

FIG. 2 illustrates an exemplary exercise database (which may correspond to exercise database 128). As illustrated, the exercise database of FIG. 2 may contain desired movement parameters for exercises that can be done with the exercise apparatus 102. These parameters may include a description of the movement, the desired movement parameters, grip type, and user adjustments. The desired movement parameters may include thresholds of movement that must be met, such as greater than two feet of vertical movement in a tall kneeling press. They may also include thresholds of movement that indicate the user is doing the exercise improperly. For example, the user would want to do the press vertically with minimal lateral movement in a tall kneeling press. In one embodiment, the user may receive haptic feedback if they move more than one inch laterally while doing a tall kneeling press. Some embodiments may include user adjustments that may be related to the user's size, strength, or fitness level. For example, the minimum vertical movement for a tall kneeling press is two feet for a user who is six feet tall. The minimum vertical movement may be one and one-half feet for a user who is only five feet tall and two feet and three inches for a user who is six feet and six inches tall. Other user adjustments may include but are not limited to movement speed, or the number of repetitions in a set may be adjusted for a user's fitness level or goals.

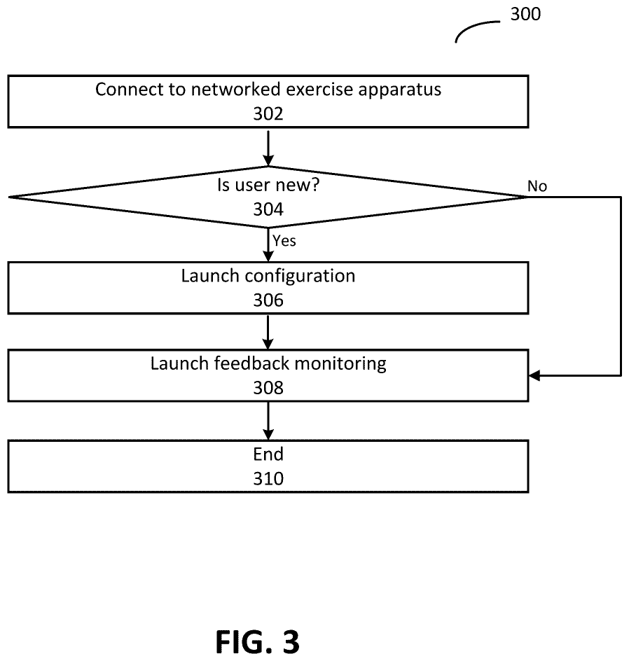

FIG. 3 is a flowchart illustrating an exemplary method 300 for exercise analytics, which may be performed in accordance with processor execution of instructions associated with exercise analytics engine 130. The method 300 of FIG. 3 may be embodied as executable instructions in a non-transitory computer readable storage medium including but not limited to a CD, DVD, or non-volatile memory such as a hard drive. The instructions of the storage medium may be executed by a processor (or processors) to cause various hardware components of a computing device hosting or otherwise accessing the storage medium to effectuate the method. The steps identified in FIG. 3 (and the order thereof) are exemplary and may include various alternatives, equivalents, or derivations thereof including but not limited to the order of execution of the

The method 300 may begin with the exercise apparatus 102 connecting to the motion network server 124 at step 302. This connection and the initialization of the exercise analytics engine 130 may be done in a number of ways. In one embodiment, the user has an app on their smartphone that connects to the exercise apparatus 102 and the motion network server 124 when the user logs in to the app. A pressure sensor in the base 104 may prompt a connection, or the base 104 or the handle 114 may have a switch that powers on the exercise apparatus 102 and prompts a connection to the motion network server 124.

Once the exercise apparatus 102 is connected to the motion network server 124, it may be determined if a new user connects to the system at step 304. If a new user is connected or wants to update their information, the configuration module 132 is launched at step 306. Once the configuration module 132 is complete or skipped, a feedback module 134 is launched at step 308. Once the user's exercise program is completed, the program ends at step 310.

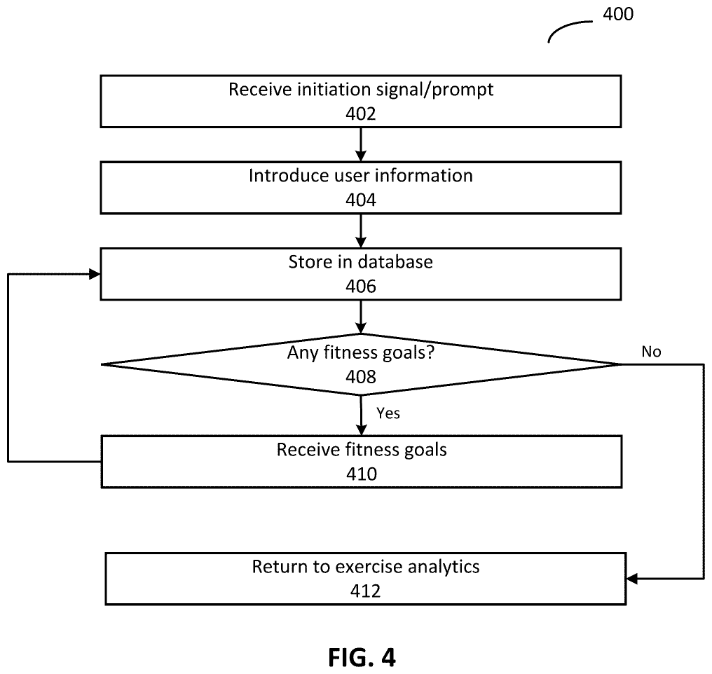

FIG. 4 is a flowchart illustrating an exemplary method 400 for apparatus configuration, which may be performed based on execution of instructions associated with configuration module 132. Method 400 may begin with receiving an initiation signal or prompt from the exercise analytics engine 130 at step 402. Such signal may request addition of a new user to the motion network server 124. The request may also pertain to an existing user who wants to update their information.

In one embodiment, user information intake and configuration of their account may be performed on an app on the user device 138. In another embodiment, the exercise apparatus 102 may collect the information directly from the user, such as with a natural language processor and a virtual assistant. User information collected may include information related to the user's build, such as height, weight, etc. Information related to the user's account may include information related to subscriptions, payment methods, devices to connect, etc.

User information may be received at step 404. Connected device information may include the configuration of sensors to collect physiological data on the user during a workout. For example, the user may have a smartwatch capable of tracking heart rate or respiration rate. That device may be paired with the system to track the user's fitness and/or their progress towards fitness goals. The information may then be written to the user database 126 at step 406.

In step 408, it may then be determined if the user wants to enter any fitness goals. Fitness goals may include but are not limited to frequency and duration of workouts, the exercises in a given workout, physiological goals such as heart or respiration rate peak, average, duration, weight loss targets, etc. For example, the user may indicate they plan to work out with the exercise apparatus 102 three times a week for thirty minutes. The user may also indicate their target heart rate of 180 beats per minute, sustained for 10 minutes.

This information regarding fitness goals may be received at step 408. The received fitness goals may then be written to the user database 126 as the method 400 returns to step 410. Once user information and fitness goals have been collected and no other new goals remain to be input, the method 400 may return to the exercise analytics engine 130 at step 412.

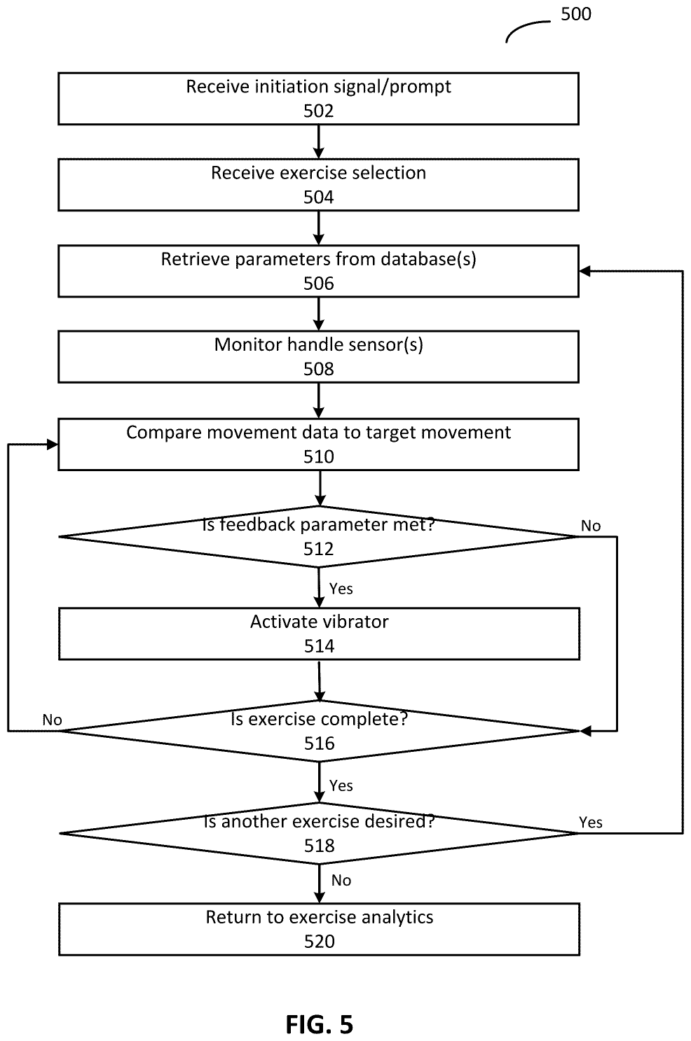

FIG. 5 is a flowchart illustrating an exemplary method 500 for feedback-based exercise management, which may be performed in accordance with execution of instructions associated with feedback module 134. Method 500 may begin with receiving an initiation signal or prompt from the exercise analytics engine 130 at step 502. An exercise selection may then be received from the user at step 504. User parameters, such as fitness goals and build information, may be retrieved from the user database 126 at step 506. The exercise selection may be received in many ways. The user may select an exercise on an app on their user device 138 from a menu of available exercises. Natural language processing may be used to identify an exercise selection made verbally by the user. In one embodiment, the user input a workout program through the configuration module 132 that included the exercises to be done, the number of reps of each exercise, and the order of the exercises to be done. For example, the user may have an exercise routine stored in the user database 126 that begins with three sets of ten tall kneeling presses. Based upon the received exercise selection, the exercise parameters may be retrieved from the exercise database 128 at step 506.

The handle sensor 116 may be monitored at step 508. In some embodiments, the base sensor 108 may be monitored instead of, or in conjunction with the handle sensor 116. Some embodiments may include multiple handle sensors 116 to monitor multiple parameters about the user, including motion, physiological parameters, and engagement metrics. Sensors in a user device 138, such as a smartwatch, may be incorporated in some embodiments. In one embodiment, the user will press an actuator in the handle 114 to initiate monitoring of an inertial movement sensor that consists of a 3-axis accelerometer, 3-axis gyroscope, and 3-axis magnetometer as the handle sensor 116. Raw accelerometer and gyroscope data must be processed to remove bias from other factors, such as gravity. A low pass filter is one of many methods known in the art for processing inertial movement sensor data into motion data.

The sensor data may be compared to the desired movements at step 510. In one embodiment, the base sensor 108 may be a camera programmed to track an indicator marking on the handle sensor 116 to monitor the user's movements and compare them to the desired movements. It may determine if the observed sensor data has reached the threshold at which feedback should be given to the user at step 512. For example, it may be observed through either the handle sensor 116 or the base sensor 108, or a combination of the two, that the handle 114 moved twenty-two inches vertically before the handle 114 begins to descend. The exercise parameters for a tall kneeling press call for twenty-four inches of vertical movement.

This observed movement may prompt the activation of the vibrator 120 at step 514 to communicate to the user that they did not complete the necessary vertical lift of the handle as prescribed by the exercise. In one embodiment, the user has provided their height as five feet and two inches tall. By comparing the height recorded in the user database 126 with the user adjustment parameter in the exercise database 128, which indicates to adjust the vertical movement by 0.5″ for every 1″ above or below 6′0″ in user height. For a user who is 5′2″, the vertical lift expected in a tall kneeling press would decrease by four inches, so that the user would be expected to achieve a minimum of twenty inches of vertical lift. In that example, the 5′2″ user executed the tall kneeling press inside the bounds of the desired movement parameters when customized to account for the user's height. This observed movement would not activate the vibrator 120.

In one embodiment, a user has specified a target heart rate of 170 beats per minute in their fitness goals in the user database 126. A heart rate sensor in the handle (handle sensor 116) may be monitored, and feedback is given when the user reaches their target heart rate. Users may specify how feedback is given. A user struggling to reach their target heart rate may elect to have feedback given through the handle 114 when the target is reached, whereas a user who is regularly reaching their target heart rate may elect to receive feedback when their heart rate drops below the target.

It may then be determined if the exercise is complete at step 516. If the exercise is not complete, the process returns to step 510. If it is determined that the exercise is completed, it may then be determined if there are more exercises to be completed in the current workout at step 518. The determination that another exercise is to be done may be based on a workout plan in the user database 126. For example, the user database 126 may indicate that after the user completes three sets of ten tall kneeling presses, they will do three sets of ten goblet squats. In that example, upon detecting the completion of the tenth tall kneeling press in the third set, the method 500 may return to step 506 to retrieve the desired movement parameters related to a goblet squat from the exercise database 128. The user may be prompted to select another exercise or end their workout in one embodiment. If the user has indicated no additional exercises, method 500 may return to the exercise analytics engine 130 at step 520.

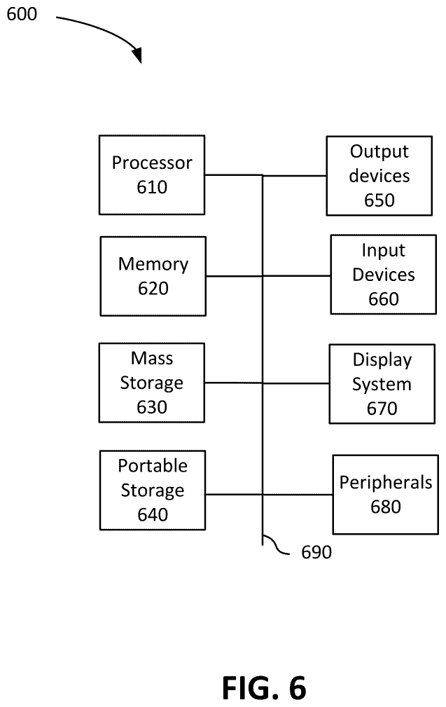

FIG. 6 illustrates an exemplary computing system 600 that may be used to implement an embodiment of the present invention. The computing system 600 of FIG. 6 includes one or more processors 610 and memory 620. Main memory 620 stores, in part, instructions and data for execution by processor 610. Main memory 620 can store the executable code when in operation. The system 600 of FIG. 6 further includes a mass storage device 630, portable storage medium drive(s) 640, output devices 650, user input devices 660, a graphics display 670, and peripheral devices 680.

The components shown in FIG. 6 are depicted as being connected via a single bus 690. However, the components may be connected through one or more data transport means. For example, processor unit 610 and main memory 620 may be connected via a local microprocessor bus, and the mass storage device 630, peripheral device(s) 680, portable storage device 640, and display system 670 may be connected via one or more input/output (I/O) buses.

Mass storage device 630, which may be implemented with a magnetic disk drive or an optical disk drive, is a non-volatile storage device for storing data and instructions for use by processor unit 610. Mass storage device 630 can store the system software for implementing embodiments of the present invention for purposes of loading that software into main memory 620.

Portable storage device 640 operates in conjunction with a portable non-volatile storage medium, such as a floppy disk, compact disk or Digital video disc, to input and output data and code to and from the computer system 600 of FIG. 6. The system software for implementing embodiments of the present invention may be stored on such a portable medium and input to the computer system 600 via the portable storage device 640.

Input devices 660 provide a portion of a user interface. Input devices 660 may include an alpha-numeric keypad, such as a keyboard, for inputting alpha-numeric and other information, or a pointing device, such as a mouse, a trackball, stylus, or cursor direction keys. Additionally, the system 600 as shown in FIG. 6 includes output devices 650. Examples of suitable output devices include speakers, printers, network interfaces, and monitors.

Display system 670 may include a liquid crystal display (LCD) or other suitable display device. Display system 670 receives textual and graphical information, and processes the information for output to the display device.

Peripherals 680 may include any type of computer support device to add additional functionality to the computer system. For example, peripheral device(s) 680 may include a modem or a router.

The components contained in the computer system 600 of FIG. 6 are those typically found in computer systems that may be suitable for use with embodiments of the present invention and are intended to represent a broad category of such computer components that are well known in the art. Thus, the computer system 600 of FIG. 6 can be a personal computer, hand held computing device, telephone, mobile computing device, workstation, server, minicomputer, mainframe computer, or any other computing device. The computer can also include different bus configurations, networked platforms, multi-processor platforms, etc. Various operating systems can be used including Unix, Linux, Windows, Macintosh OS, Palm OS, and other suitable operating systems.

The functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

What is claimed is:

1. An apparatus for providing feedback accuracy of exercise movement, the apparatus comprising;
a ball;
a stick extending from the ball; and
one or more sensors associated with the ball, each of the sensors configured to measure a characteristic of movement of the ball.

2. The apparatus of claim 1, further comprising a base that includes at least a concave region having at least a partial spherical region configured to hold the ball.

3. The apparatus of claim 1, further comprising a handle at a distal end of the stick, wherein the handle is rotatable relative to the stick.

4. The apparatus of claim 3, further comprising one or more handle sensors configured to measure a characteristic of movement of the handle.

5. The apparatus of claim 1, further comprising a radio configured to transmit data regarding the measured characteristic to one or more designated devices over a communication network.

6. A system for providing feedback accuracy of exercise movement, the system comprising;
an exercise apparatus that includes a ball, a stick extending from the ball, and one or more sensors associated with the ball, wherein each of the sensors is configured to measure a characteristic of movement of the ball; and
a motion network server that:
stores data in a database in memory regarding a plurality of different types of exercises,
receives sensor data from the exercise apparatus over a communication network regarding the measured characteristic of movement of the ball,
identifies that the data corresponds to one of the types of exercise, and
compares the received data to data stored in an exercise database regarding the identified type of exercise; and
generates feedback regarding the comparison between the received data and stored data regarding the identified type of exercise.

7. The system of claim 6, further comprising a database that stores data regarding one or more users.

8. The system of claim 7, wherein the data includes physical data, health data, fitness data, or goal data associated with one or more of the users.

9. The system of claim 7, wherein the motion network server further collects the data regarding the users.

10. The system of claim 6, wherein the feedback includes an indication of whether a movement indicated by the received data matches the stored data regarding the identified type of exercise.

11. A method for providing feedback accuracy of exercise movement, the method comprising;
storing data in a database in memory regarding a plurality of different types of exercises;
monitoring movement of a ball of an exercise apparatus that includes the ball, a stick extending from the ball, and one or more sensors associated with the ball, wherein each of the sensors is configured to measure a characteristic of the movement of the ball;
analyzing sensor data received from the sensors of the exercise apparatus over a communication network regarding the measured characteristic of movement of the ball;
identifying that the data corresponds to one of the types of exercise, and
comparing the received data to data stored in an exercise database regarding the identified type of exercise; and
generating feedback regarding the comparison between the received data and stored data regarding the identified type of exercise.

12. The method of claim 11, further comprising storing data in memory regarding one or more users.

13. The method of claim 12, wherein the data includes physical data, health data, fitness data, or goal data associated with one or more of the users.

14. The method of claim 12, further comprising collecting the data regarding the users.

15. The method of claim 11, wherein the feedback includes an indication of whether a movement indicated by the received data matches the stored data regarding the identified type of exercise.

16. The method of claim 11, further comprising customizing a learning model based on the sensor data, wherein the learning model is configured to correlate sensor data to one or more exercise parameters.

17. The method of claim 16, further comprising generating a custom exercise regimen based on the exercise parameters identified by the customized learning model.

18. The method of claim 16, wherein the learning model corresponds to data regarding an identified user of the exercise apparatus.

19. The method of claim 16, further comprising updating the customized learning model over time based on sensor data associated with subsequent exercises, and modifying the custom exercise regimen in real-time based on the updated learning model.

20. A non-transitory, computer-readable storage medium, having embodied thereon a program executable by a processor to perform a method for providing feedback accuracy of exercise movement, the method comprising;
storing data in a database in memory regarding a plurality of different types of exercises;
monitoring movement of a ball of an exercise apparatus that includes the ball, a stick extending from the ball, and one or more sensors associated with the ball, wherein each of the sensors is configured to measure a characteristic of the movement of the ball;
analyzing sensor data received from the sensors of the exercise apparatus over a communication network regarding the measured characteristic of movement of the ball;
identifying that the data corresponds to one of the types of exercise, and
comparing the received data to data stored in an exercise database regarding the identified type of exercise; and
generating feedback regarding the comparison between the received data and stored data regarding the identified type of exercise.
